(19) United States
(12) Patent Application Publication
(10) Pub. No.: US 2026/0232360 A1
(43) Pub. Date: Aug. 13, 2026

(54) POLYAXIAL SCREW ASSEMBLY AND ORTHOPEDIC COMPONENT

(71) Applicant: LIMACORPORATE S.P.A., San Daniele del Friuli (IT)

(72) Inventors: FERRO THOMAS, Mortegliano (IT); Andrea FATTORI, Cividale del Friuli (IT)

(21) Appl. No.: 19/149,775

(22) PCT Filed: Jan. 22, 2024
(86) PCT No.: PCT/EP2024/051407

§ 371 Date: Jul. 21, 2025

(30) Foreign Application Priority Data
Jan. 27, 2023 (IT) 102023000001239

(51) Int. Cl. A61B 17/80; A61B 17/86

(52) U.S. Cl. CPC A61B 17/8047; A61B 17/8605

(57) ABSTRACT

Polyaxial screw assembly (100) comprising: a base element (101) having a hole (102) that defines at least one recess (103a, 103b) on a perimeter thereof; a substantially annular-shaped bushing (201), that is inserted in the hole (102) and tiltable, comprising at least one outer protrusion (202a, 202b) fitted inside said at least one recess (103a, 103b); a screw (301) inserted in the bushing (201) and passing in the hole (102), wherein the screw (301) comprises a head (302) and a stem (303), the head (302) being configured to cooperate with the bushing (201) for an angular locking in a spatial direction of the stem (303) with respect to the hole (102). The recess (103a, 103b) has a respective groove that is open toward a top side of the base element (101) and having an extent configured for a raising or lowering of the outer protrusion (202a, 202b), allowing a first partial rotation of the bushing (201) prior to the angular locking.

### FIELD OF APPLICATION

The present invention relates to a polyaxial screw assembly and a relevant orthopedic component.

The present invention finds a particular application in the field of orthopedics, particularly applied to prostheses, especially modular prostheses such as joint prostheses, but also to bone plates of various types.

### PRIOR ART

It is known to use screws in the orthopedic field, in order to obtain a suitable fixation between implant and bone and to allow a proper bone integration.

More in detail, there are different types of screw, differing in the fixation principle:
A) Compressive screws, that are screwed in a hole of a metal component interposed between the screw head and the bone, so as to compress the metal component against the bone itself. In this case the screw is kept compressed against the implant but it is not mechanically integral therewith, allowing it to be inserted at different angles depending on the desired direction.
B) Monoaxial screws with angular stability: in that case the metal component is integral with the screw and there is not a real compression of the metal component against the bone, but precisely due to the angular stability of the screw, it is able to resist shear stresses. This typology has the disadvantage that it can be inserted only along a predetermined direction, that perhaps is not suitable in many practical cases.
C) Polyaxial screws with angular stability: these allow to insert the screw in different directions according to need and then provide them, during the screwing, with angular stability i.e. making them integral with the metal component according to a direction determined on a case-by-case basis, within a polyaxial movement range.

Document US2006/0190090A1 refers to a polyaxial screw for acetabular cup, wherein the screw passes through a hole housing a ring mounted in the shell of the acetabular cup; the ring can be rotated and it is a split ring comprising a non-circular outer profile that cooperates with the non-circular profile of the hole arranged in the shell.

Document WO2009/042511A1 refers to a polyaxial locking screw system, wherein a bone plate defines a hole with an inner spherical surface and a polyaxial bushing with a split structure having an outer spherical surface is provided inside the hole, to receive a bone screw. The outer surface of the polyaxial bushing comprises a plurality of spikes to lock the screw at the desired angle.

Document WO2013/037939A1 refers to an implant comprising an opening, an anchoring element and a locking mechanism with a radially deformable annular element which can be pressed against the head of the anchoring element to fix the head in the implant opening. The implant opening has a recess having a support surface for the head of the anchoring element.

Document WO2013/167895A1 refers to a locking mechanism for a polyaxial locking screw, comprising a bushing with a partially spherical outer surface being groove-like sized and shaped to closely conform to a cavity in a receiving element.

Document WO2009/097537A1 refers to a bone plate system with a hole for bone screw and a locking element; the plate has an opening for bone screw) defined by a side wall comprising a first and a second substantially planar segments, and the locking element is coupled to the plate and at least partially positioned in the opening. The locking element has an outer geometry that defines a first and a second substantially planar surfaces, that engage segments of the side wall of the opening, respectively, in order to prevent the relative movement between the locking element and the plate.

Document WO2015/103090A1 refers to reverse glenoid implants that include an anchoring screw and a base plate having a distal end with a first opening sized to receive a head of the anchoring screw. The anchoring screw is retained against an axial translation with respect to the base plate, but it can rotate with respect to the base plate.

Document WO2 007/050796A2 refers to a bone fixation assembly comprising a bone plate and a bushing having a hole adapted to receive a screw. The portion of the bone plate and the bushing are cooperatively configured to provide a pivoted movement of the bushing inside the hole about a joint axis and limit the joint axis to a plane that extends in the portion of the bone plate.

Prior art solutions that provide a polyaxial locking of the screw are not however fully effective and can therefore be improved.

An object of the present invention is to allow a polyaxial locking of a screw that is effective.

A further object of the present invention is to allow a polyaxial locking of a screw by using components having an alternative configuration with respect to the prior art.

A further object of the present invention is to allow a polyaxial locking of a screw inside a base element.

A further object of the present invention is to provide a polyaxial locking configuration having such structural and functional features as to overcome drawbacks of the prior art.

### SUMMARY OF THE INVENTION

A solution idea underlying the present invention is to provide a locking system for polyaxial screws with angular stability, comprising a bushing, i.e. a component that is inserted in the implant shell or plate to be stabilized, also defined as base element. The bushing receives the head of the bone screw to provide it with angular stability.

The shell or plate includes a suitable recess-like shaping, in particular a pair of recesses that are diametrically opposite with respect to the hole, to receive the bushing. The bushing is inserted by slight elastic deformation thereof into the hole, so that, once inserted, it can no longer come out.

The bushing has a partially spherical outer contour, intended to match the spherical portion of the hole. One or more side protrusions of the bushing allow to avoid a rotation thereof during the screwing, while allowing however a raising and a partial rotation along each section, so as to make the central axis of the bushing assume any direction within a conical angle, that is polyaxial.

The bushing has a plurality of radially deformable elements (preferably elastically) or “fins”, so that during the screwing of the screw head in the respective seat inside the bushing, there is an expansion of the radially deformable elements or “fins” peripherally pushing them against the spherical seat of the hole. In particular, the head cooperates with the bushing in an at least partially conical coupling to allow the angular locking.

The cooperation by friction between the surface of the radially deformable elements or “fins” against the spherical seat of the hole allows to lock the spatial position of the base element with hole/bushing/bone screw assembly.

Based on said solution idea, the present invention provides a polyaxial screw assembly.

The polyaxial screw assembly comprises a base element having a hole, the hole defining at least one recess on a perimeter of the hole itself.

The polyaxial screw assembly comprises a substantially annular-shaped bushing, inserted in the hole and tiltable with respect to an axis of the hole. The bushing comprises at least one outer protrusion, fitted inside the at least one respective recess.

The polyaxial screw assembly comprises a screw inserted in the bushing and passing in the hole. The screw comprises a head and a stem; the head is configured to cooperate with the bushing for an angular locking in a spatial direction of the stem with respect to the hole.

The at least one recess has a respective groove that is open toward a top side of the base element.

The groove has an extent configured for a raising or lowering of the at least one outer protrusion, allowing a first partial rotation of the bushing prior to the locking thereof.

Advantageously, the polyaxial screw assembly according to the present invention allows a more effective polyaxial locking of the screw with respect to the base element.

Preferably, there is a pair of recesses and a respective pair of protrusions, in which a first protrusion is raised and a second protrusion is lowered during the first partial rotation of the bushing; in fact, the pair of outer protrusions is configured to slide inside the two grooves, respectively.

Preferably, the at least one recess and the respective groove are further configured for a second partial rotation of the bushing prior to the polyaxial locking of the screw, thus allowing the bushing to pivot around the pair of outer protrusions.

Preferably, the at least one protrusion and the at least one recess are further configured to prevent a third partial rotation of the bushing about an axis of the hole, during a screwing of the screw inside the bushing.

Preferably, the hole in the base element defines an at least partially spherical side wall thereof, and the bushing comprises a respective partially spherical outer contour. In this way, a shape coupling is provided between the side wall and the contour outer, to allow the polyaxial locking by friction of the bushing.

Preferably, the bushing has an annular shape with a closed perimeter and comprises a plurality of partial slots extending along an insertion direction of the screw. The partial slots define a plurality of radially deformable portions, for a friction interaction of the bushing with the base element and the screw.

Moreover, the present invention refers to an orthopedic component, such as a prosthesis element or a bone plate, that comprises at least one polyaxial screw assembly according to the present invention.

Further features and advantages of the invention will be apparent from the following detailed description of embodiments, given by way of non-limiting example, and from the claims that form an integral part of the present description.

### BRIEF DESCRIPTION OF THE DRAWINGS

FIG. 1 illustrates an example of an orthopedic component equipped with a polyaxial locking screw according to the prior art.

FIG. 2 shows an embodiment of a polyaxial screw assembly in an assembled configuration.

FIG. 3 shows the polyaxial screw assembly of FIG. 2 in a disassembled configuration.

FIG. 4 shows a bushing partially inserted in a base element, in an embodiment of the present invention.

FIG. 5 shows a bushing completely inserted in a base element, in an embodiment of the present invention

FIG. 6 Shows a View in a First Section of FIG. 5.

FIGS. 7A, 7B, 7C, 7D show views in a second section of FIG. 5, in which the bushing is rotated with respect to the base element

FIG. 8 shows the partial insertion of a screw inside the bushing with respect to the configuration of FIG. 7A.

FIG. 9 shows the complete insertion of the screw of FIG. 8.

FIG. 10 shows the complete insertion of a screw inside the bushing with respect to the configuration of FIG. 6, in which the bushing is partially rotated.

FIG. 11 shows a second embodiment of a polyaxial screw assembly in a disassembled configuration.

FIGS. 12A and 12B show sectional views of the bushing and of the base element corresponding to the assembly of FIG. 11, in an assembled configuration.

In different figures, analogous elements will be indicated by analogous reference numbers. If several analogous elements are present in a single figure, sometimes only one or some of them will be indicated with a respective reference number for a better readability, meaning that the other ones are also encompassed in the disclosure.

### DETAILED DESCRIPTION

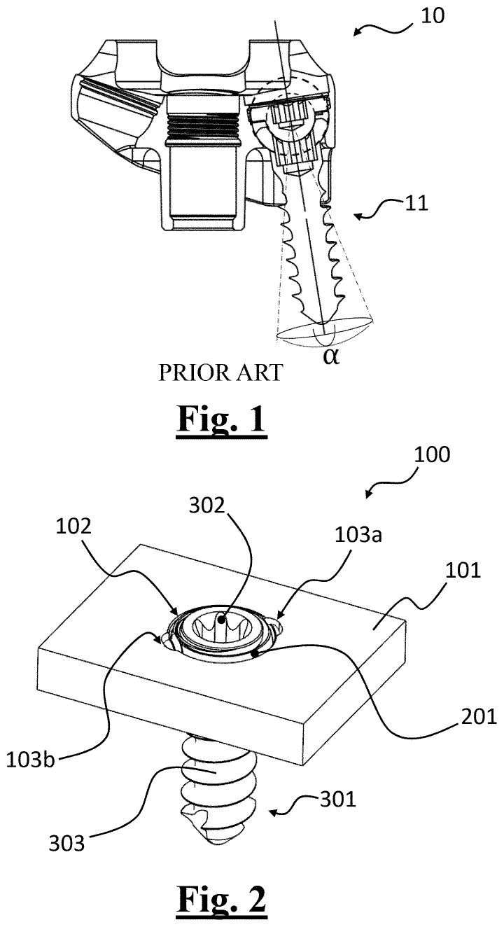

FIG. 1 illustrates an example of orthopedic component 10, in particular a scapular component of a reverse shoulder prosthesis.

The orthopedic component 10 is equipped with a polyaxial locking screw 11, according to the prior art. The polyaxial locking screw 11 allows to define a direction within the conical angle a and then to lock this angular position for implanting the screw in the bone.

The introduction of screws 11 with angular stability has been a key improvement in surgical technique, that has helped in improving the fixation of the orthopedic component (in this example, also referred to as “baseplate”) in terms of reduction of possible relative movements with respect to the bone. In other words, the adoption of screws 11 with angular stability and polyaxial locking reduces the possibility of implant failure, and it is thus preferred by many surgeons.

FIG. 2 shows a first exemplary embodiment of the present invention, providing a polyaxial screw assembly 100 in an assembled configuration. The polyaxial screw assembly 100 comprises a base element 101 having a hole 102. The hole 102 defines at least one recess on the perimeter thereof, in particular a pair of recesses 103a and 103b, that are diametrically opposite to each other in the hole 102, that will be further described.

The polyaxial screw assembly 100 comprises a bushing 201 that is inserted in the hole 102, as it will be further described.

The polyaxial screw assembly 100 comprises a screw 301, inserted in the bushing 201 and passing in the hole 102, that will be further described.

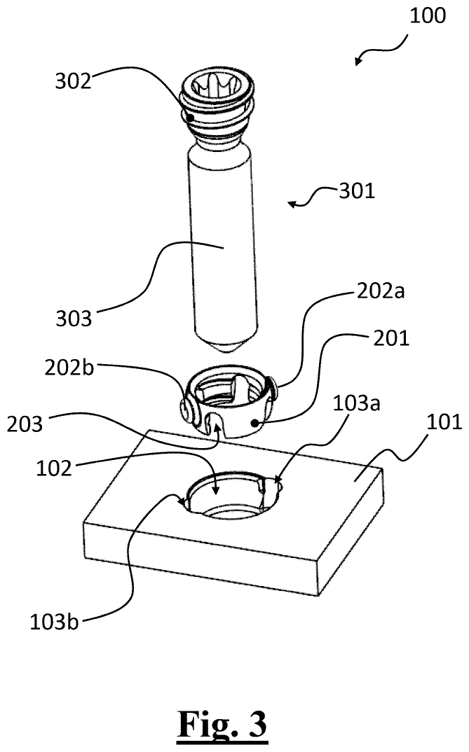

FIG. 3 shows the polyaxial screw assembly 100 in a disassembled configuration, for a better understanding thereof.

The base element 101 has the hole 102 that defines the pair of diametrically opposite recesses 103a and 103b. This base element 101 in this example precisely simulates an implant part to be stabilized by the application of one (or more) screws with angular stability. Possible applications to orthopedic components 101 such as prosthesis elements or bone plates will be listed hereinafter.

The recesses 103a and 103b have two respective grooves that, as it can also be seen, are open toward a top side of the base element 101.

In particular, the grooves of the recesses 103a and 130b have a substantially rectilinear development, that is more particularly free of localized enlargements and/or constrictions.

As it can be understood by considering also FIG. 2, the side defined as “top” of the base element 101, where the two grooves of the recesses 103a and 103b are open, is a side of the base element 101 that is opposite to a final mounting position of the stem 303 of the screw 301.

The bushing 201 has a substantially annular shape and is insertable into the hole 102. Moreover, as it will be further described, the bushing 201 is tiltable with respect to an axis of the hole 102.

In particular, the bushing 201 has an annular shape with a closed perimeter and further comprises a plurality of partial slots 203, that extend along an insertion direction of the screw 301 inside the bushing 201.

The plurality of partial slots 203 define a plurality of radially deformable portions of the bushing 201 that, as it will be further described, allow a friction interaction of the bushing 201 with the base element 101 and with the screw 301.

The bushing 201 comprises at least one outer protrusion, in particular a pair of outer protrusions 202a and 202b that are diametrically opposite to each other in the bushing 201. This pair of outer protrusions 202a and 202b is fitted inside the pair of respective recesses 103a and 103b.

The pair of outer protrusions 202a and 202b comprises two respective button-like protrusions, having each a substantially cylindrical body and radially protruding from an annular body of the bushing 201.

The screw 301 is in turn inserted in the bushing 201 and thus is passing in the hole 102. The screw 301 comprises a head 302 and a stem 303 (that, in this representation, is modelled for simplicity as devoid of threading, unlike what is shown in FIG. 2).

The head 302 of the screw 301 is configured in particular to cooperate with the bushing 201, for an angular locking in a spatial direction of the stem 303 with respect to the hole 102, as it will be better described.

The stem 303 of the screw 301 is the portion of the assembly 100 intended to be screwed in the bone, and that is to be provided with angular stability by polyaxial locking.

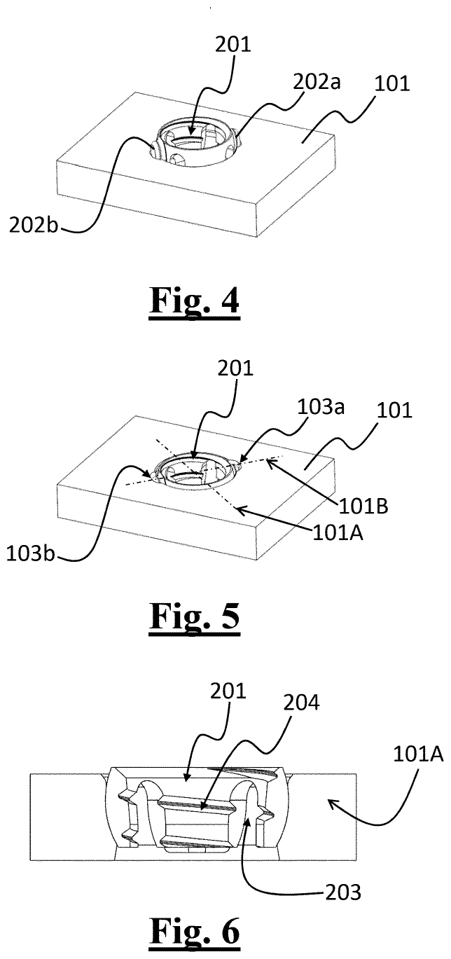

FIG. 4 shows the bushing 201 partially inserted in the base element 101, while FIG. 5 shows the same bushing 201 completely inserted in the same base element 101.

It can be understood how the two grooves 103a and 103b are configured for the passage of the pair of outer protrusions 202a and 202b, so as to allow insertion of the bushing 201 into the hole 102.

In particular, the bushing 201 is insertable un-angled and coaxial with respect to the axis (the vertical one, in this example) of the hole 102.

FIG. 6 shows a view in a first section 101A indicated by the line of FIG. 5.

In this sectional view it can be understood that the bushing 201 is at least partially elastically deformable, for insertion into the hole 102. In this way, the bushing 201 is retained after insertion thereof in the hole 102, and the bushing 201 is prevented from accidentally getting out of the hole 102 before the screw 301 is inserted.

In particular, as already described, the bushing 201 comprises a plurality of partial slots 203 that extend along an insertion direction of the screw 301 and define a plurality of radially deformable portions of the bushing 201. In this way, a friction interaction of the bushing 201 with the base element 101 and the screw 301 can be improved.

Moreover, preferably, the bushing 201 further comprises an inner contour 204 that is threaded and whose object will be further described.

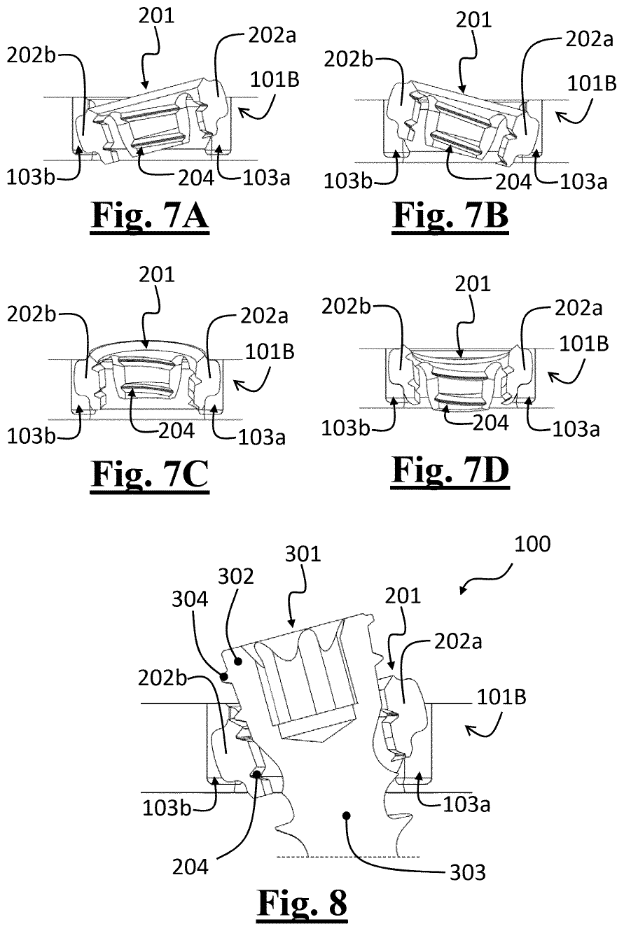

FIGS. 7A, 7B, 7C, 7D show views in a second section 101B indicated by the line of FIG. 5. In said views, the bushing 201 is rotated with respect to the base element 101, precisely because the bushing 201 allows a polyaxial orientation of the screw 301 with respect to the base element 101.

In general, the two grooves 103a and 103b have an extent configured to allow a raising or lowering, respectively, of each of the outer protrusions 202a and 202b, as shown. In this way, a first partial rotation, in particular in the plane of the section 101B, of the bushing 201 is allowed prior to an angular locking of the screw 301.

Detailing the solution, the bushing 201 has an annular shape partially modelled as a sphere cut above the center line, allowing insertion of the bushing 201 by elastic deformation thereof into the hole 102. Once it is inserted, the bushing 201 cannot accidentally come out, unless it is elastically deformed once again.

Once the bushing 201 is inserted, the sphere-on-sphere coupling with the corresponding partially spherical seat of the hole 102 is exploited so that the bushing 201 can rotate about the center of the sphere. The central axis of the bushing 201 can thus be directed in any direction within a conical angle.

Preferably, the two grooves 103a and 103b have a depth extent inside the base element 101 that is deeper than a neutral position of the pair of outer protrusions, as it can be seen for example in FIGS. 7C and 7D. The “neutral” position is defined by the bushing not being tilted with respect to the axis of the hole 102, for example as shown also in FIG. 5.

Preferably, a protrusion of the pair of outer protrusions 202a and 202b is partially jutting above a respective one of the two grooves 103a and 103b, in a maximally tilted position. The “maximally tilted” position is defined by the bushing being maximally tilted with respect to the axis of the hole 102, for example as shown in FIGS. 7A and 7B.

In particular, in FIG. 7A the outer protrusion 202a is partially jutting above the respective groove 103a, while the outer protrusion 202b is inserted near the bottom of the respective groove 103b.

Preferably, in the first partial rotation of the bushing 201, a first protrusion 202a is raised while a second protrusion 202b is lowered, as in the example of FIG. 7A, or vice versa as in the example of FIG. 7B. In general, the pair of outer protrusions 202a and 202b is configured to slide inside the grooves 103a and 103b, respectively to allow the first rotation of the bushing 201, that allows in turn the polyaxial orientation of the screw fitted therein.

Moreover, as in the examples represented in FIGS. 7C and 7D, the two grooves 103a and 103b are further configured for a second partial rotation of the bushing 201, prior to the angular locking.

In particular, in the second rotation the bushing 201 is allowed to pivot around the pair of outer protrusions 103a and 103b, tilting on a plane that is perpendicular to that of the first partial rotation, i.e. aligned with the line 101B of FIG. 5.

Moreover, the first partial rotation (for example, in the plane of the line 101B of FIG. 5) and the second partial rotation (for example, in the plane of the line 101A of FIG. 5) can be composed with each other, resulting in an overall rotation that defines, for the stem of the screw inserted in the bushing 201, a conical angle in the space.

FIG. 8 shows the partial insertion of the screw 301 inside the bushing 201, in a configuration that is comparable to that of FIG. 7A.

In this view it can be appreciated that the head 302 of the screw 301 comprises a threading 304, precisely configured to cooperate in screwing with the inner contour 204 of the bushing 201, being it too threaded correspondingly to the threading 304.

Moreover, the head 302 cooperates with the bushing 201 in an at least partially conical coupling between the surfaces.

In this way, the head 302 cooperates in screwing with the bushing 201, allowing a more effective angular locking.

In general, the conical shape of the head 302 of the screw 301 helps to elastically enlarge the bushing 201, allowing the angular locking as it will be further described.

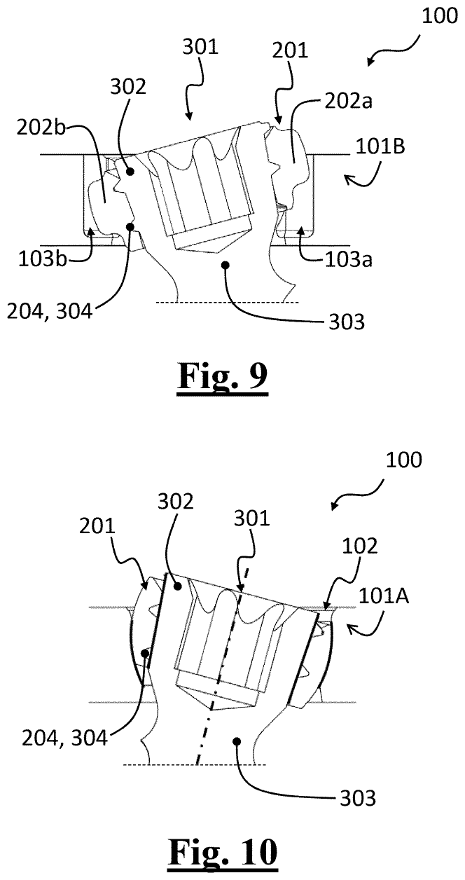

FIG. 9 shows the complete insertion of the screw 301 inside the bushing 201, in a configuration that is still comparable to that of FIG. 7A and to that of FIG. 8.

It can be understood how, during a screwing of the screw 301 inside the bushing 201 engaging a bone by means of the stem 303, the pair of outer protrusions 202a and 202b and the pair of recesses 103a and 103b are further configured to prevent a third partial rotation of the bushing 201, about an axis of the hole 102 itself.

In other words, the outer protrusions 202a and 202b and the recesses 103a and 103b cooperate to lock a torsional rotation of the bushing 201 inside the hole 102, easing the assembling and the polyaxial locking of the screw.

FIG. 10 shows the complete insertion of the screw 301 inside the bushing 201, in a configuration that is comparable to that of FIG. 6, but in which the bushing 201 is partially rotated according to the second partial rotation.

In this view, obtained on a section along the line 101A of FIG. 5, it can

be appreciated that the hole 102 of the base element 101 defines an at least partially spherical side wall thereof. Moreover, the bushing 201 comprises a partially spherical outer contour, configured for a shape coupling with the side wall of the hole 102.

The shape coupling, by the friction between the bushing 201 and the side wall of the hole 102 of the base element, allows the angular locking of the stem 303 of the screw 301.

Obviously, in this section along the line 101A the recesses and the outer protrusions of the bushing 201 cannot be seen.

Further detailing the solution, the screw 301 is inserted along any direction among those allowed within the conical angle and, in the insertion step, it aligns the bushing 201 in the direction chosen by the surgeon.

Then, the screw 301 is screwed in the bone until the head 302 of the screw 301 comes into contact with the bushing 201.

Afterwards, a screwing of the threading 304 of the head 302 of the screw 301 with the respective threaded seat 204 inside the bushing 201 occurs.

From then on, by keeping on screwing the screw 301, the conical coupling between the head 302 and the seat in the bushing 201 makes sure that there is a force component that peripherally pushes the fins of the bushing 201 against the spherical seat of the hole 102, locking by friction the position of the bushing 201 assembled with the screw 301 in the chosen direction.

The outer protrusions 202a and 202b and the respective recesses 103a and 103b, in the step of tightening the screw 301, prevent the bushing 201 from slipping inside the spherical surface of the hole 102.

In this way, the tightening torque of the screw 301 is sufficient to generate a friction that locks the position of the axis of the screw 301, providing the assembly with angular stability.

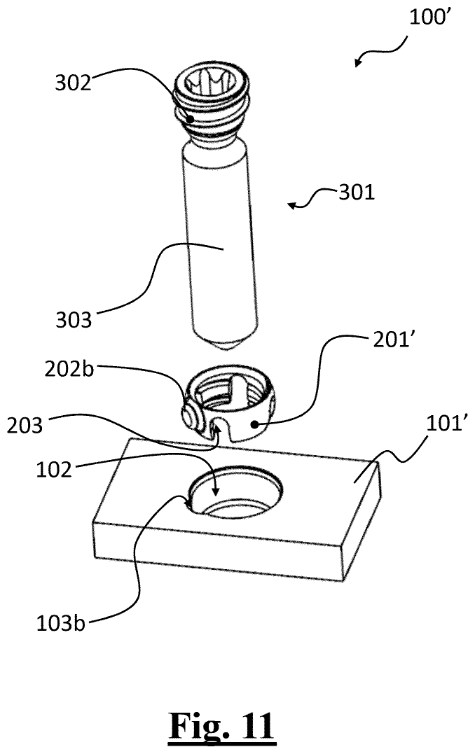

FIG. 11 shows a second exemplary embodiment of the present invention, of a polyaxial screw assembly 100′ in a disassembled configuration.

The polyaxial screw assembly 100′ comprises a base element 101′ having a hole 102 that, with respect to the above-exemplified embodiment, only comprises one recess on the perimeter thereof, in particular the recess 103b.

The polyaxial screw assembly 100′ comprises a bushing 201′ that is inserted in the hole 102, and a screw 301 inserted in the bushing 201′ and that is similar to the already described one.

The single recess 103b and generally the polyaxial screw assembly 100′ substantially correspond to what has already been described above with reference to FIG. 3, except that in this case the second recess 103a is absent.

The bushing 201′ comprises an outer protrusion 202b fitted inside the respective recess 103b.

In this case too, the head 302 of the screw 301 is configured in particular to cooperate with the bushing 201′, for an angular locking in a spatial direction of the stem 303 with respect to the hole 102, as it will be better described.

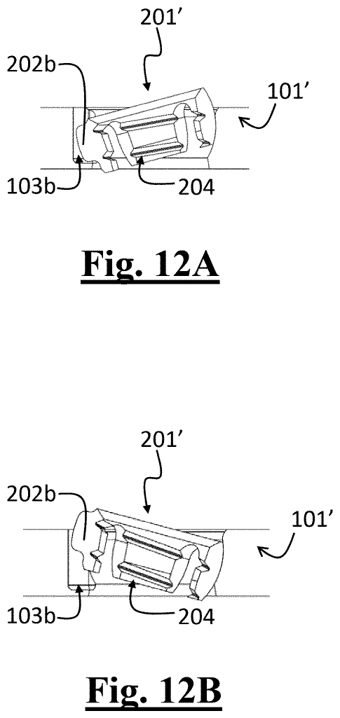

FIGS. 12A and 12B show sectional views of the bushing 201′ and of the relevant base element 101′corresponding to the polyaxial screw assembly 100′ in an assembled configuration. These sectional views are comparable to those of the above-described FIGS. 7A and 7B, that are along the second section 101B.

The bushing 201′ is rotated with respect to the base element 101′, precisely because the bushing 201′ allows a polyaxial orientation of the screw 301 with respect to the base element 101′ as well.

In general, the groove 103b has an extent configured to allow a raising or lowering, respectively, of the outer protrusion 202b, as shown in the two views.

In this way, even with a single groove and a respective outer protrusion, the first partial rotation of the bushing 2011 prior to an angular locking of the screw 301 is still allowed.

The bushing 201′, similarly to the already-described bushing 201, has an annular shape partially modelled as a sphere cut above the center line as well, allowing insertion of the bushing 201′ by elastic deformation thereof into the hole 102 and directing the central axis thereof within a conical angle.

In general, the outer protrusion 202b is configured to slide inside the groove 103b to allow the first rotation of the bushing 201′, that allows in turn the polyaxial orientation of the screw fitted therein.

Similarly to what has already been described for the polyaxial screw assembly 100, during a screwing of the screw 301 inside the bushing 201′ engaging a bone by means of the stem 303, the outer protrusion 202b and the recess 103b are further configured to prevent a third partial rotation of the bushing 201′, about an axis of the hole 102 itself.

It is thus evident that having a single recess 103b and a respective protrusion 202b is effective in providing a locking for polyaxial screws with angular stability, similarly to having a pair of recesses 103a and 103b and a respective pair of protrusions 202a and 202b.

In the case of plates 101′ having a very reduced thickness, a high tilt of the bushing 201′ could cause the single protrusion 202b to come out during the screwing of the screw 301 in the bushing 201′. In order to prevent this drawback, that does not occur instead in the case of the plates 101 and bushing 201, the plate 101′ can be slightly modified in a variant (not represented), locally raising the edges of the plate 101′ in the area that is adjacent to the recess 103b, with a localized increase in the thickness of the base element 101′.

### INDUSTRIAL APPLICABILITY

The polyaxial screw assembly of the present invention can be applied to different areas and for any type of bone screw.

In general an orthopedic component, such as a prosthesis element or a bone plate, provides at least one hole for the passage of a polyaxial screw. The hole defines at least one recess, preferably a pair of diametrically opposite recesses, with at least one respective groove, preferably two respective grooves that are open toward a top side.

The hole is configured to receive a respective bushing and a respective screw, according to a polyaxial screw assembly of the present invention.

In particular, several applications are listed below by way of example:
A. Shoulder prosthesis, comprising polyaxial screws to fix a baseplate to the glenoid. Preferably two of the four holes that are present on the implantable component are used.
B. Hip prosthesis. Polyaxial screws can be used to fix the “augments” by exploiting the screw direction that is considered the most appropriate by the surgeon, and then the implant cup can be inserted.
C. Knee prosthesis.
D. Osteosynthesis plates. Polyaxial screws can be used to fix osteosynthesis plates.

It is evident that further implementations and modifications of the present invention will be possible for the person skilled in the art, in order to meet contingent requirements.

For example, the conformation of the bushing and of the screw head can be adjusted based on the hole diameter and on the overall dimensions of the assembly. Very small holes will require equally reduced bushings and screws, with simplified geometries. Larger holes will allow to adopt bushings and screws having a greater diameter, with more elaborate geometries.

The examples of the present description are thus to be understood as merely illustrative but not limiting.

1. A polyaxial screw assembly (100) comprising:
a base element (101) having a hole (102), said hole (102) defining at least one recess (103a, 103b) on a perimeter of said hole (102);
a substantially annular-shaped bushing (201), inserted in said hole (102) and tiltable with respect to an axis of said hole (102), said bushing (201) comprising at least one outer protrusion (202a, 202b) fitted inside said at least one recess (103a, 103b);
a screw (301) inserted in said bushing (201) and passing in said hole (102), wherein said screw (301) comprises a head (302) and a stem (303), said head (302) being configured to cooperate with said bushing (201) for an angular locking in a spatial direction of said stem (303) with respect to said hole (102); wherein said at least one recess (103a, 103b) has a respective groove that is open toward a top side of said base element (101), and wherein said groove has an extent configured for a raising or lowering of said at least one outer protrusion (202a, 202b), allowing a first partial rotation of said bushing (201) prior to said angular locking.

2. The polyaxial screw assembly according to claim 1, wherein said groove of said at least one recess (103a, 103b) has a depth extent inside said base element (101) being deeper than a neutral position of said at least one outer protrusion (202a, 202b), said bushing (201) being not tilted with respect to said axis of said hole (102) in said neutral position.

3. The polyaxial screw assembly according to claim 1, wherein said at least one outer protrusion (202a, 202b) is partially jutting above said respective groove of said at least one recess (103a, 103b) in a maximally tilted position of said bushing (201) with respect to said axis of said hole (102).

4. The polyaxial screw assembly according to claim 1, wherein said groove of said at least one recess (103a, 103b) has a substantially rectilinear development, being free from localized enlargements and/or constrictions.

5. The polyaxial screw assembly according to claim 1, wherein said at least one recess (103a, 103b) comprises a pair of diametrically opposite recesses (103a, 103b) in said hole (102), and wherein said at least one outer protrusion (202a, 202b) comprises a pair of diametrically opposite outer protrusions (202a, 202b) in said bushing (201), said pair of outer protrusions (202a, 202b) being fitted inside said pair of recesses (103a, 103b).

6. The polyaxial screw assembly according to claim 5, wherein a first protrusion (202a) of said pair of outer protrusions is raised and a second protrusion (202a) of said pair of outer protrusions is lowered in said first partial rotation of said bushing (201), said pair of outer protrusions (202a, 202b) being configured to slide inside two grooves of said pair of recesses (103a, 103b), respectively.

7. The polyaxial screw assembly according to claim 1, wherein said groove of said at least one recess (103a, 103b) is further configured for the passage of said at least one outer protrusion (202a, 202b) and for insertion of said bushing (201) into said hole (102), said bushing (201) being insertable un-angled and coaxial with respect to said axis of said hole (102).

8. The polyaxial screw assembly according to claim 7, wherein said bushing (201) is at least partially elastically deformable, for insertion into said hole (102) so as to prevent said bushing (201) from getting out of said hole (102) prior to an insertion of said screw (301).

9. The polyaxial screw assembly according to claim 1, wherein said at least one outer protrusion (202a, 202b) comprises a button-like protrusion, having a substantially cylindrical body radially protruding from an annular body of said bushing (201).

10. The polyaxial screw assembly according to claim 1, wherein said groove of said at least one recess (103a, 103b) is further configured for a second partial rotation of said bushing (201) prior to said angular locking, allowing said bushing (201) to pivot about said at least one outer protrusion (202a, 202b).

11. The polyaxial screw assembly according to claim 10, wherein said at least one outer protrusion (202a, 202b) and said at least one recess (103a, 103b) are further configured to prevent a third partial rotation of said bushing (201) about an axis of said hole (102) during a screwing of said screw (301).

12. The polyaxial screw assembly according to claim 1, wherein said top side, where said groove of said at least one recess (103a, 103b) is open, is a side of said base element (101) being opposite to a final mounting position of said stem (303) of said screw (301).

13. The polyaxial screw assembly according to claim 1, wherein said hole (102) defines an at least partially spherical side wall thereof, and wherein said bushing (201) comprises a partially spherical outer contour configured for a shape coupling with said side wall to allow said angular locking by friction.

14. The polyaxial screw assembly according to claim 1, wherein said head (302) of said screw (301) comprises a threading, and wherein said bushing (201) further comprises an inner contour, said inner contour being threaded correspondingly to said head (302), said head (302) cooperating with said bushing (201) in an at least partially conical coupling to allow said angular locking.

15. The polyaxial screw assembly according to claim 1, wherein said bushing (201) has an annular shape with a closed perimeter and further comprises a plurality of partial slots (203) extending along an insertion direction of said screw (301), said plurality of partial slots (203) defining a plurality of radially deformable portions of said bushing (201), said radially deformable portions of said bushing (201) opening outward with insertion of said screw (301) and providing a friction interaction with said base element (101).

16. An Orthopedic component, such as a prosthesis element or a bone plate, said orthopedic component having at least one hole (102), said at least one hole (102) defining at least one recess (103a, 103b) on a perimeter of said at least one hole (102), wherein said at least one recess (103a, 103b) has a respective groove being open toward a top side of said base element (101), characterized in that said at least one hole (102) is configured to receive a respective bushing (201) and a respective screw (301) of a polyaxial screw assembly according to claim 1.
